(19) United States
(12) Patent Application Publication
(10) Pub. No.: US 2026/0337673 A1
(43) Pub. Date: Oct. 29, 2026

(54) A METHOD OF DETECTING THE PRESENCE OF A NUCLEIC ACID

(71) Applicant: Agency for Science, Technology and Research, Singapore (SG)

(72) Inventors: Ichiro Hirao, Singapore (SG); Michiko Hirao, Singapore (SG)

(21) Appl. No.: 19/107,759

(22) PCT Filed: Aug. 31, 2023
(86) PCT No.: PCT/SG2023/050602

§ 371 Date: Feb. 28, 2025

(30) Foreign Application Priority Data
Aug. 31, 2022 (SG) 10202250869F

(51) Int. Cl. C12Q 1/686; C12Q 1/48; C12Q 1/6827; C12Q 1/6851; C12Q 1/6853; C12Q 1/6876

(52) U.S. Cl. CPC C12Q 1/686; C12Q 1/48; C12Q 1/6827; C12Q 1/6851; C12Q 1/6853; C12Q 1/6876; C12Y 207/07; C12Q 2600/106; C12Q 2600/156; G01N 2800/00

(57) ABSTRACT

There is provided a method detecting the presence of a nucleic acid in a sample, the method comprises performing an amplification reaction on the sample in the presence of a proofreading polymerase and a primer with a 2′-sugar modification. Also provided is a method of identifying a disease in subject and a kit for detecting a nucleic acid in a sample.

### RELATED APPLICATIONS

This application is the U.S. National Stage of International Application No. PCT/SG2023/050602, filed Aug. 31, 2023, which designates the U.S., published in English, and claims priority under 35 U.S.C. § 119 or 365 (c) to Singapore Application No. 10202250869F, filed Aug. 31, 2022. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 5975.1032-001 Corrected Sequence Listing.xml; created May 14, 2025, 29,011 Bytes in size.

### TECHNICAL FIELD

The present disclosure relates broadly to a method of detecting the presence of a nucleic acid in a sample.

### BACKGROUND

qPCR is becoming a powerful tool for the detection and quantification of single nucleotide polymorphisms (SNPs), somatic mutations, and infectious disease subtypes of deoxyribonucleic acid (DNA) and ribonucleic acid (RNA) targets. qPCR has the ability to monitor the amplification of a target nucleic acid during the PCR in real time and can be used quantitatively. Two common methods for the detection of PCR products in qPCR are i) non-specific fluorescent dyes that intercalate with any double-stranded DNA and ii) sequence specific DNA probes consisting of oligonucleotides that are labelled with a fluorescent reporter, which permits detection only after hybridization of the probe with its complementary sequence. Quantitative polymerase chain reaction (qPCR) using SYBR Green detection provides an easy and inexpensive method that can be optimised to cater an allelic discrimination assay.

Despite the advantages of qPCR, to enable sensitive detection of SNPs, somatic mutations or small aberrations in nucleic acid, high fidelity PCR amplification is necessary. It has been found that the use of proofreading DNA polymerases typically provides high fidelity PCR amplification. However, due to the digestion of PCR primer by the exonuclease activity, the proofreading DNA polymerases cannot be used for the conventional SNP detection by qPCR using match- and mismatch-primers. Furthermore, potential drawbacks include high cost for probe-based qPCR and low specificity for intercalating dye-based qPCR.

Therefore, there is a need to provide an alternative method to detect the presence of nucleic acid (such as SNP) in a sample.

### SUMMARY

In one aspect, there is provided a method of detecting the presence of a nucleic acid in a sample,
the method comprises performing an amplification reaction on the sample in the presence of a proofreading polymerase and a primer with a 2′-sugar modification.

In some examples, the proofreading polymerase is an exonuclease proficient polymerase.

In some examples, the proofreading polymerase is a 3′-exonuclease proficient polymerase, optionally a 3′-exonuclease proficient DNA polymerase.

In some examples, the 2′-sugar modification is at locations in the primer comprising the 5′ end of the primer, the middle of the primer, or the 3′ end of the primer.

In some examples, the 2′-sugar modification is at the 3′ end of the primer.

In some examples, the 2′-sugar modification is at a position that is two residues from the 3′ end of the primer.

In some examples, the 2′-sugar modification is at penultimate position of the 3′ end of the primer.

In some examples, the primers with 2′-sugar modification comprises 2′-O-methyl-ribonucleosides modification or 2′-fluoro.

In some examples, the 2′-sugar modification is one modification selected from the group consisting of 2′-O-methyl C primer/C_{OME}, 2′-O-methyl T primer/T_{OME}, 2′-O-methyl U primer/U_{OME}, 2′-O-methyl G primer/G_{OME}, and 2′-O-methyl A primer/A_{OME}.

In some examples, the primer comprises 15 to 30 nucleic acid residues.

In some examples, the method comprises detecting the presence of a nucleotide mismatch or nucleotide polymorphism.

In some examples, the method further comprises the step of quantification of the nucleic acid detected in the sample.

In some examples, the method comprises a method of genome detection for therapeutics, authentication methods, or visual PCR detection.

In another aspect, there is provided a method of identifying a disease in a subject, comprising detecting the presence of a nucleic acid fragment by performing an amplification reaction on a sample obtained from the subject in the presence of a proofreading polymerase and a primer with a 2′-sugar modification.

In some examples, the nucleic acid fragment/template comprises a sequence selected from the group listed in Table 2 below or a part thereof.

In some examples, the primers comprise a sequence selected from the group listed in Table 3 below or a part thereof.

In some examples, the sample is a biological sample or a clinical sample.

In some examples, the method further comprises a step of quantifying the target nucleic acid.

In yet another aspect, there is provided a kit for detecting a nucleic acid in a sample, the kit comprising one or more of the following: one or more primers/primer set comprising a primer with 2′-sugar modification, and a proofreading polymerase.

### DESCRIPTION OF EMBODIMENTS

Quantitative PCR (qPCR) allows multiple research applications such as gene expression analysis, genotyping, allelic discrimination, single nucleotide polymorphism (SNP) detection, microbe and pathogen detection, or species abundance quantification. qPCR determines the actual amount of PCR product present at a given cycle by using a fluorescent reporter in the PCR reaction to measure DNA generation in the qPCR assay.

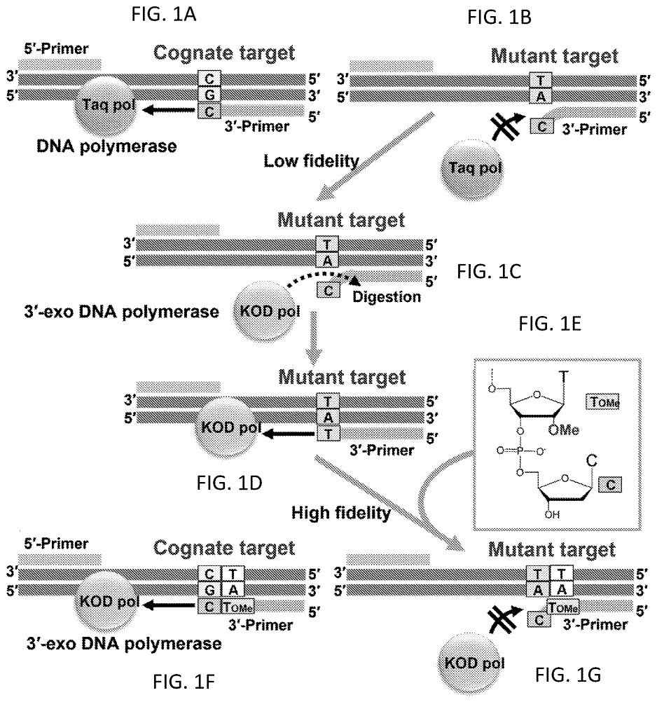

The representative SNP detection method is qPCR using allele-specific primers that anneal with their 3′-ends at the SNP site, in which an additional mismatch at one of the last five nucleotides from the 3′-end increases the fidelity (Amplification Refractory Mutation System, ARMS). Matched primers with their template DNAs proceed with primer extension (FIG. 1A). In contrast, mismatched primers at the 3′-ends of the templates prevent the primer extension (FIG. 1B). However, the destabilizing effects of some mismatched pairs, such as G-T and A-C, are weak and result in false positive results. In addition, the design of effective primers for ARMS-PCR depends on the target sequence context. To detect somatic mutations in a DNA pool, allele-specific qPCR requires the quantification of a population frequency less than 1%.

Conventional allele-specific qPCR employs DNA polymerases without the 3′→5′ exonuclease proofreading function (exo-DNA polymerases), such as Taq DNA polymerase. Although 3′-exonuclease-proficient (exo+) proofreading DNA polymerases possess high fidelity in replication, the exo+ nuclease activities digest the mismatched nucleotide at the 3′-ends in the allele-specific primers, allowing the extension of the mismatched primers (FIGS. 1C and 1D). Due to the digestion of PCR primer by the exonuclease activity, the proofreading DNA polymerases cannot be used for the conventional SNP detection by qPCR using match-and mismatch-primers. Furthermore, current qPCR methods in the art are high in cost with low specificity.

As used herein, the term “DNA polymerase” refers to a type of enzyme that catalyzes the formation of new DNA strands by adding nucleotides to the existing DNA template strand. The enzyme also plays a role in repairing damaged DNA. The function of the DNA polymerase is essential for passing on genetic information. The 3′→5′ exonuclease activity intrinsic to several DNA polymerases plays a primary role in genetic stability; it acts as a first line of defense in correcting DNA polymerase errors. The 5′→3′ exonuclease activity can be coupled to the polymerization activity to displace DNA strands/to remove ribonucleotide primers that are used in DNA replication.

As such, the inventors of the present disclosure provide alternative methods to detect the presence of nucleic acid in a sample.

In one aspect, there is provided a method of detecting/identifying/determining the presence of a nucleic acid in a sample, the method comprises performing an amplification reaction on the sample in the presence of a proofreading polymerase and a primer with a 2′-sugar modification.

As used herein, the term “nucleic acid” refers to a nucleotide sequence that typically includes nucleotides comprising an A, G, C, T or U base. In some examples, nucleotide sequences may include other bases such as inosine, methylcytosine, hydroxymethylcytosine, methylinosine, methyladenosie and/or thiouridine, and the like. The term “nucleic acid” may include both single and/or double stranded deoxyribonucleic acid (DNA) and ribonucleic acid (RNA), including environmental DNA (eDNA), genomic DNA, bacterial DNA, viral DNA, cell-free DNA (cfDNA), complementary RNA (cRNA), messenger RNA (mRNA), transfer RNA (TRNA), microRNA (miRNA), cell-free RNA (cfRNA), circulating tumor RNA (ctRNA), bacterial RNA, viral RNA, ribosomal RNA (rRNA), and the like.

In some examples, the nucleic acid may be a DNA or an RNA. In some examples, the DNA may include but is not limited to, a cell-free DNA, a circulating DNA, a circulating cell-free DNA, and the like. In some examples, the RNA may include but is not limited to, a cell-free RNA, a circulating RNA, a circulating cell-free RNA, and the like. In some examples, the method includes a cell-free DNA.

Advantageously, the use of the combination of modified primers and proofreading polymerase significantly improves the fidelity/sensitivity of the amplification method (such as quantitative polymerase chain reaction (qPCR)) to detect a nucleic acid as compared to other modifications known in the art. The method of the present disclosure has higher sensitivity than the use of primers with locked nucleic acid (LNA) or 2′-fluoro modifications. The experimental data in the present disclosure shows that LNA method detects about 0.04% population frequency, and 2′-fluoro modification detects about 5% population frequency, which are both less sensitive than the O-methyl-ribonuclease modifications of detecting about 0.01% to 0.2% population frequency of target mutations in a DNA. Furthermore, the current O-methyl-ribonuclease modification method is simple and cost effective compared to other modifications known in the art (such as LNA modifications) which are expensive.

In some examples, the method comprises an amplification reaction with an eIongation time of 0.5 minutes, 1 minutes, 1.5 minutes, 2 minutes, 2.5 minutes, 3 minutes, 3.5 minutes, 4 minutes, 4.5 minutes, 5 minutes, 5.5 minutes, or 6 minutes or more. In some examples, the method includes an eIongation time of 3.5 minutes in the amplification reaction.

In various embodiments, the proofreading polymerase as used herein is an exonuclease proficient polymerase.

As used herein, the term ‘proofreading polymerase’ refers to a polymerase with a proofreading domain, such as a 3′-exonuclease proficient polymerase. A proofreading polymerase checks each nucleotide during DNA synthesis and excise mismatched nucleotides in the 3′ to 5′ direction. The proofreading activity allows removal of the mismatched nucleotide from the primer before it is extended and is important to correct errors during replication.

In various embodiments, the proofreading polymerase is a 3′-exonuclease proficient polymerase, optionally a 3′-exonuclease proficient DNA polymerase.

In some examples, the 3′-exonuclease proficient DNA polymerase may include, but is not limited to, KODplus DNA polymerase, Pfu polymerase, Vent polymerase, Klenow fragment, T7 DNA polymerase, and the like.

Without wishing to be bound by theory, the inventors of the present disclosure found that 3′-exonuclease proficient DNA polymerase provides better detection of nucleic acid as compared to the use of 3′ exonuclease deficient DNA polymerase.

Without wishing to be bound by theory, it is believed that proofreading DNA polymerase with 3′-exonuclease activity provides high-fidelity PCR amplification. However, proofreading DNA polymerase cannot be used for conventional SNP detection by qPCR using matched and mismatched primers as the exonuclease activity digests the PCR primer with a mismatched base. To overcome this problem, the inventors of the present disclosure have found that use of modified primers (such as those with 2′-sugar modification) advantageously prevents digestion of the PCR primer by the exonuclease activity of the proofreading polymerase. This allows high fidelity detection by amplification reactions, with cognate targets being amplified with the matched primers, and mutant targets with minimal amplification signal compared to the cognate targets.

In various embodiments, the 2′-sugar modification as disclosed herein is at locations in the primer such as but is not limited to the 5′ end of the primer, the middle of the primer, the 3′ end of the primer, and the like.

In various embodiments, the 2′-sugar modification is at 3′ end of the primer.

In various embodiment, the 2′-sugar modification is at a position that is two residues from the 3′ end of the primer.

In some examples, the 2′-sugar modification is on the second last residue from the 3′ end of the primer.

In some examples, the primer may comprise one or more 2′-sugar modification. In some examples, the primer may comprise 1, 2, 3, or more 2′-sugar modification.

In various embodiments, the 2′-sugar modification is at penultimate position of the 3′ end of the primer.

As used herein, the term “penultimate” refers to a position that is 2 residues from the 3′ end of the primer, or the second last residue from the 3′ end of the primer.

In some examples, the primer with 2′-sugar modification may be designed to target each strand of a double stranded nucleic acid.

In various embodiments, the primers with 2′-sugar modification may comprise, but is not limited to, 2′-O-methyl-ribonucleosides modification, 2′-fluoro, and the like.

In some examples, the primers may comprise locked nucleic acid (LNA) modification.

In various embodiments, the 2′-sugar modification may include one or more modification such as, but is not limited to, 2′-O-methyl C primer/C_{OME}, 2′-O-methyl T primer/T_{OME}, 2′-O-methyl U primer/U_{OME}, 2′-O-methyl G primer/G_{OME}, and 2′-O-methyl A primer/A_{OME}, and the like.

Here, the inventors of the present disclosure present a simple on/off switch method of high-fidelity qPCR method using the combination of proofreading DNA polymerases (such as 3′-exonuclease-proficient (exo⁺) proofreading DNA polymerases, as exemplified by KODplus DNA polymerase in FIG. 1E to 1G) and a simple 2′-sugar modified nucleoside.

Advantageously, the modification as disclosed herein increase the stability of the primers against 3′ exonuclease activity of high-fidelity polymerases, resulting in enhanced fidelity as compared to that of the conventional method using exonuclease-deficient DNA polymerases.

In addition, the inventors have also observed that the modification as disclosed herein significantly reduce primer dimer production during the amplification reaction. The 2′-O-methyl-modified primers efficiently prevent primer dimer production in PCR.

The method as disclosed herein significantly improved the qPCR fidelity and can quantitatively detect a 0.01-0.2% population frequency of target mutations in a DNA sample. The method of the present disclosure was capable of quantitatively detected the EGFR T790M mutation in circulating tumor DNA isolated from clinical blood samples.

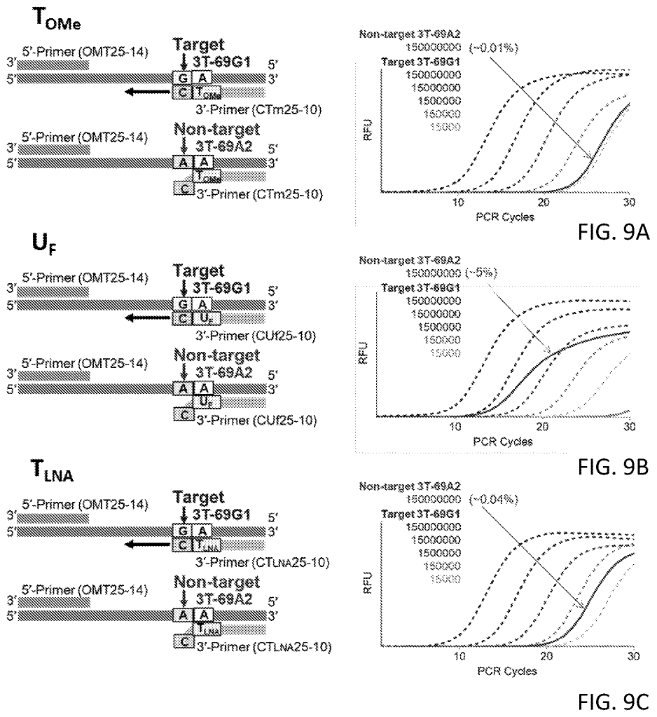

To increase the sensitivity and specificity of allele-specific qPCR by using exo⁺ DNA polymerases, several qPCR platforms, such as 3′-labeled exo⁺ primer-extension assays and on/off or off/on switch assays, have been reported. Among them, the on/off switch assay using primers containing a locked nucleic acid (LNA) exhibits highly specific allele detection. Introducing an LNA residue to the penultimate position from the 3′-ends of allele-specific primers effectively prevents the digestion by exo⁺ DNA polymerases, and thus is applicable to allele-specific qPCR for detecting somatic mutations. The inventors of the present disclosure also examined qPCR with the primer using LNA. As compared the LNA system, the method of the present disclosure using O-methyl exhibited higher sensitivity: the method of the present disclosure can detect ~0.01% population frequency of target mutations in a DNA and the LNA method detects ~0.04% population frequency (FIG. 9). In addition, the LNA primer is expensive, and the 2′-O-methyl primer is more cost effective.

The method of the present disclosure with the combination of 2′-O-methyl modified primers and proof-read DNA polymerase is a high-fidelity allele specific qPCR method with simple, cost effective, and highly specific for SNP detection. 2′-O-Methyl-ribonucleosides at the penultimate position from the 3′-ends of primers completely prevent the digestion by the exonuclease activity of exo⁺ DNA polymerases and can identify a 0.01-0.2% population frequency of target mutations in a DNA pool, which is as high as that using LNA-primers.

In various embodiments, the primer may comprise 15 to 30 nucleic acid residues.

In some examples, the primers may be about 15 to 30 residues in length. In some examples, the primers may be about, but is not limited to, 15, 16, 17, 18, 19, 20, 21, 22, 23, 24, 25, 26, 27, 28, 29, or 30 residues, and the like.

In some examples, the primer may comprise 18 to 25 nucleic acid residues. In some examples, the primer may comprise 18 residues.

In some examples, the primer may comprise more than one modification. For example, the primer may further comprise a locked nucleic acid modification. In some examples, the primer may not comprise locked nucleic acid modification.

In some examples, the primers may be provided using methods known in the art. In some examples, the primers may be chemically synthesized. In some examples, the primers may be a commercially available primer that has been modified using methods as described herein. In some examples, the primers are modified by having a 2′-sugar modification at the 3′ end, and to target each strand of the nucleic acid. In some examples, the modified primers are validated for PCR efficiency and specificity.

In some examples, the method comprises the use of 0.1 μM, 0.2 μM, 0.3 μM, 0.4 μM, 0.5 μM, 0.6 μM, 0.7 μM, 0.8 μM, 0.9 μM, 1 μM, 1.1 μM, 1.2 μM, 1.3 μM, 1.4 μM, or 1.5 μM or more of modified primers. In some examples, the method comprises the use of 0.3 μM of modified primers.

In some examples, the method may further comprise providing a probe.

In some examples, the probe may comprise 15 to 30 nucleic acid residues.

In some examples, the probe may be about 15 to 30 residues in length. In some examples, the probe may be about, but is not limited to, 15, 16, 17, 18, 19, 20, 21, 22, 23, 24, 25, 26, 27, 28, 29, or 30 residues, and the like.

In some examples, the probe may comprise 18 to 25 nucleic acid residues. In some examples, the probe may comprise 18 residues.

In some examples, the probe may comprise more than one modification. For example, the probe may further comprise a locked nucleic acid modification. In some examples, the probe may not comprise locked nucleic acid modification.

In some examples, the probes may be provided using methods known in the art. In some examples, the probes may be chemically synthesized. In some examples, the probes may be a commercially available probe that has been modified using methods as described herein. In some examples, the probes are modified by having a 2′-sugar modification at the 3′ end, and to target each strand of the nucleic acid. In some examples, the modified probes are validated for PCR efficiency and specificity.

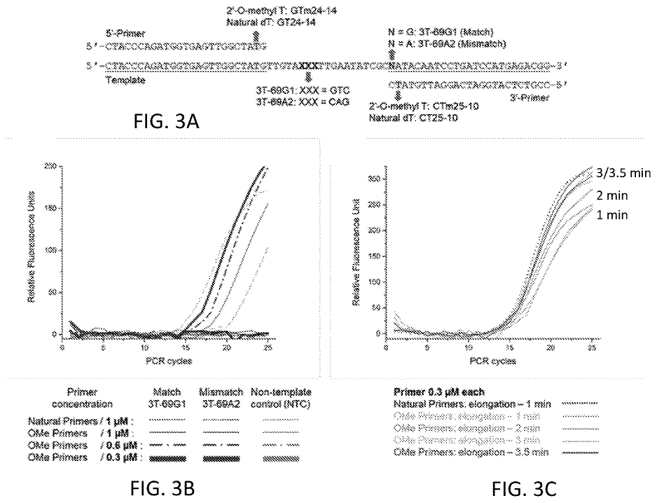

As shown in FIG. 3 to FIG. 10, the inventors of the present disclosure showed that the method as disclosed herein can be used to detect or differentiate different targets, such as matched target/target, mismatched target/non target, non-template control (NTC), and the like. As such, in some examples, the method may comprise one or more template for the amplification reaction. In some examples, the one or more template for the amplification reaction may comprise a template for a matched target/target, a template for a mismatched target/non target, and/or a non-template control (NTC).

As used herein, the term “template” refers to a nucleic acid fragment with or without a nucleotide mismatch or single nucleotide polymorphism. In some examples, a primer/probe anneals to a template to allow subsequent steps of amplification reaction to occur. In some examples, the template is a synthesized nucleic acid fragment. In some examples, the synthesized nucleic acid fragment is produced by known in the art methods. In some examples, the synthesized nucleic acid fragment includes ligating a nucleic acid fragment (such as in the presence of Splint DNA to guide the ligation) to an existing nucleic acid fragment. In some examples, the synthesized nucleic acid is purified after ligation of a nucleic acid fragment to an existing nucleic acid fragment.

In some examples, the nucleic acid fragment/template may be assayed for purity. As shown in the experimental section, the inventors of the present disclosure could show that, with barcode sequencing, the nucleic acid fragment/template were able to be chemically synthesized with around 99.9% purity. The purity of the nucleic acid fragment/template may be determined using methods known in the art. For example, the purity can be detected using Ion PGM sequencing. In some examples, the fragments that are used for nucleic acid fragment/template purity analysis may include, but is not limited to, one or more of the fragments listed in Table 1 below or a part thereof.

TABLE 1

| Fragments for | |
|---|---|
| DNA purity | |
| analysis | Sequence (5′ to 3′) |
| BacoreAdaptor49 | CTCACTACGCNNNNNNNNNNNNNNCTTCGTTG |
| | AGAACCCACAATCTTAC |
| | (SEQ ID NO: 1) |
| Splint20 | GCGTAGTGAGCCGTCTCATG |
| | (SEQ ID NO: 2) |
| Primer 25mer | GTAAGATTGTGGGTTCTCAACGAAG |
| | (SEQ ID NO: 3) |

In various embodiments, the method detects/identifies/determines the presence of a nucleotide mismatch/nucleotide polymorphism.

In various embodiments, the method detects/identifies/determines the presence of a single nucleotide polymorphism (SNP).

In various embodiments, the method further comprises the step of quantification of the nucleic acid detected in the sample.

In some examples, the nucleic acid detected in the sample may be quantified by methods known in the art such as but is not limited to quantitative real-time PCR, next generation sequencing, UV absorbance with spectrophotometer, fluorescence dyes, agarose gel electrophoresis, microfluidic capillary electrophoresis, diphenylamine method, droplet digital PCR, and the like.

In various embodiments, the method may be a method of genome detection for therapeutics (such as cancer diagnostics, or pathogen mutation/variation detection in infectious diseases, or genetic defect detection, or allele-specific detection), or authentication methods (such as using biometric DNA ink), or visual PCR detection (such as for amplified DNA using a genetic alphabet expansion system, or for antibiotic resistant bacteria e.g., quinoIone resistant bacteria), and the like.

In some examples, the method of detecting/identifying/determining a nucleic acid for cancer diagnostics may comprise, but is not limited to, thyroid cancer, pancreatic cancer, breast cancer, coIon cancer, lung cancer, liver cancer, skin cancer, and the like.

In some examples, the method of detecting/identifying/determining a nucleic acid for pathogen mutation/variant detection in infectious diseases may comprise a nucleic acid of a bacterial pathogen, a viral pathogen, a fungal pathogen, or a parasite.

Examples of a bacterial pathogen may include, but is not limited to, Escherichia coli, Group B streptococci, vancomycin-resistant enterococci (VRE), Acetobacter aurantius, Acinetobacter baumannii, Actinomyces Israelii, Agrobacterium radiobacter, Agrobacterium tumefaciens, Azorhizobium caulinodans, Azotobacter vinelandii, Anaplasma phagocytophilum, Anaplasma marginale, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaminogenicus (Prevotella melaminogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia complex, Burkholderia cenocepacia, Calymmatobacterium granulomatis, Campylobacter coli, Campylobacter fetus, Campylobacter jejuni, Campylobacter pylori, Chlamydia trachomatis, Chlamydophila. (such as C. pneumoniae, Chlamydophila psittaci, Clostridium botulinum, Clostridium difficile, Clostridium perfringens, Clostridium tetani), Corynebacterium diphtheriae, Corynebacterium fusiforme, Coxiella bumetii, Ehrlichia chaffeensis, Enterobacter cloacae, Enterococcus avium, Enterococcus durans, Enterococcus faecalis, Enterococcus 1 Enterococcus galllinarum, Enterobacter gergoviae (now known as Pluralibacter gergoviae), Enterococcus maloratus, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis Peptostreptococcus, Porphyromonas gingivalis, Pseudomonas aeruginosa, Pseudomonas syringae, Rhizobium Radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella spp (such as Salmonella enteritidis, Salmonella typhi, Salmonella typhimurium, and the like), Serratia marcescens, Shigella dysenteriae, Staphylococcus spp (such as Staphylococcus aureus, Staphylococcus epidermidis, and the like), Stenotrophomonas maltophilia, Streptococcus agalactiae, Streptococcus. avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis and Yersinia pseudotuberculosis.

Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Fusarium oxysporum, Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae and Arthroderma vanbreuseghemii, Aspergillus flavus, Aspergillus fumigatus and Aspergillus niger, Blastomyces dermatitidis, Candida albicans, Candida glabrata, Candida guilliermondii, Candida krusei, Candida parapsilosis, Candida tropicalis and Candida pelliculosa, Cladophialophora carrionii, Coccidioides immitis and Coccidioides posadasii, Cryptococcus neoformans, Cunninghamella Sp, Epidermophyton floccosum, Exophiala dermatitidis, Filobasidiella neoformans, Fonsecaea pedrosoi, Fusarium solani, Geotrichum candidum, Histoplasma capsulatum, Hortaea werneckii, Issatschenkia orientalis, Madurella grisae, Malassezia furfur, Malassezia globosa, Malassezia obtusa, Malassezia pachydermatis, Malassezia restricta, Malassezia slooffiae, Malassezia sympodialis, Microsporum canis, Microsporum fulvum, Microsporum gypseum, Microsporidia, Mucor circinelloides, Nectria haematococca, Paecilomyces variotii, Paracoccidioides brasiliensis, Penicillium marneffei, Pichia anomala, Pichia guilliermondii, Pneumocystis jiroveci, Pneumocystis carinii, Pseudallescheria boydii, Rhizopus oryzae, Rhodotorula rubra, Scedosporium apiospermum, Schizophyllum commune, Sporothnx schenckii, Trichophyton mentagrophytes, Trichophyton rubrum, Trichophyton verrucosum and Trichophyton violaceum, and Trichosporon asahii, Trichosporon cutaneum, Trichosporon inkin, Trichosporon mucoides, and the like.

Examples of a parasite may include, but is not limited to, Leishmania parasites, Giardia, Cryptosporidium, Entamoeba and the like.

In some examples, the genetic defects may include, but is not limited to, a prenatal genetic defect, Cystic fibrosis, and the like. In some examples, the prenatal genetic defect may include, but is not limited to, Down syndrome (Trisomy 21), Turner Syndrome, Edwards' syndrome, and the like.

In another aspect, there is provided a method of identifying a disease in a subject, comprising detecting/determining the presence of a nucleic acid fragment by performing an amplification reaction on a sample obtained from the subject in the presence of a proofreading polymerase and a primer with a 2′-sugar modification.

In various embodiments, the nucleic acid fragment/template as disclosed herein may comprise a sequence selected from the group listed in Table 2 below or a part thereof.

TABLE 2

| Templates | |
|---|---|
| for PCR | Sequence (5′ to 3′) |
| 3T-69G1 | CTACCCAGATGGTGAGTTGGCTATGTTGTAGTCTTGAC |
| | TATCGCGATACAATCCTGATCCATGAGACGG |
| | (SEQ ID NO: 4) |
| 3T-69A2 | CTACCCAGATGGTGAGTTGGCTATGTTGTACAGTTGAC |
| | TATCGCAATACAATCCTGATCCATGAGACGG |
| | (SEQ ID NO: 5) |
| 69(1) | CTACCCAGATGGTGAGTTGGCTATGTTGTAGTCTTGAA |
| | TATCGCGTTACAATCCTGATCCATGAGACGG |
| | (SEQ ID NO: 6) |
| 69(2) | CTACCCAGATGGTGAGTTGGCTATGTTGTAGTCTTGAA |
| | TATCGCATTACAATCCTGATCCATGAGACGG |
| | (SEQ ID NO: 7) |
| 69(3) | CTACCCAGATGGTGAGTTGGCTATGTTGTAGTCTTGAA |
| | TATCGCGCTACAATCCTGATCCATGAGACGG |
| | (SEQ ID NO: 8) |
| 69(4) | CTACCCAGATGGTGAGTTGGCTATGTTGTAGTCTTGAA |
| | TATCGCACTACAATCCTGATCCATGAGACGG |
| | (SEQ ID NO: 9) |
| 69(5) | CTACCCAGATGGTGAGTTGGCTATGTTGTAGTCTTGAA |
| | TATCGCGGTACAATCCTGATCCATGAGACGG |
| | (SEQ ID NO: 10) |
| 69(6) | CTACCCAGATGGTGAGTTGGCTATGTTGTAGTCTTGAA |
| | TATCGCAGTACAATCCTGATCCATGAGACGG |
| | (SEQ ID NO: 11) |

In various embodiments, there is provided a method the primers comprise a sequence selected from the group listed in Table 3 below or a part thereof.

TABLE 3

| | Sequence (5′ to 3′) |
|---|---|
| | t = 2′-O-methyl T (OMeT), a = 2′-O-methyl |
| | A (OMeA), g = 2′-O-methyl G |
| | (OMeG), c = 2′-O-methyl C (OMeC), u = |
| Primers | 2′-O-methyl U (OMeU), I = locked |
| for PCR | nucleic acid T (LNA-T), f = 2′-fluoro-U |
| OMT25- | CCGTCTCATGGATCAGGATTGTAtC (SEQ ID NO: 12) |
| 10 | |
| OMA25- | CCGTCTCATGGATCAGGATTGTAaC (SEQ ID NO: 13) |
| 10 | |
| OMG25- | CCGTCTCATGGATCAGGATTGTAgC (SEQ ID NO: 14) |
| 10 | |
| OMC25- | CCGTCTCATGGATCAGGATTGTAcC (SEQ ID NO: 15) |
| 10 | |
| T25-10 | CCGTCTCATGGATCAGGATTGTATC (SEQ ID NO: 16) |
| OMU25- | CCGTCTCATGGATCAGGATTGTAuC (SEQ ID NO: 17) |
| 10 | |
| LT25-10 | CCGTCTCATGGATCAGGATTGTAIC (SEQ ID NO: 18) |
| FU25-10 | CCGTCTCATGGATCAGGATTGTAfC (SEQ ID NO: 19) |
| TOMT25- | CCGTCTCATGGATCAGGATTGTATT (SEQ ID NO: 20) |
| 10 | |
| TT25-10 | CCGTCTCATGGATCAGGATTGTATT (SEQ ID NO: 21) |
| OMT25- | CTACCCAGATGGTGAGTTGGCTAtG (SEQ ID NO: 22) |
| 14 | |
| T25-14 | CTACCCAGATGGTGAGTTGGCTATG (SEQ ID NO: 23) |
| OMU25- | CTACCCAGATGGTGAGTTGGCTAuG (SEQ ID NO: 24) |
| 14 | |
| LT25-14 | CTACCCAGATGGTGAGTTGGCTAIG (SEQ ID NO: 25) |
| FU25-14 | CTACCCAGATGGTGAGTTGGCTAfG (SEQ ID NO: 26) |

In various embodiments, there is provided a method wherein the sample is a biological sample or a clinical sample.

In some examples, the biological sample/clinical sample may comprise a solid sample or a liquid/fluid sample. In some examples, the liquid/fluid sample may comprise a blood sample.

As used herein, the term “biological sample” refers to a sample obtained from a biological subject, including a sample of biological tissue or fluid origin obtained in vivo or in vitro. Hence, a “biological sample” may be a solid biological sample or a liquid biological sample. Examples of a “solid biological sample” may include biopsies, such as an organ biopsy, a tumor biopsy, stools, cell culture, food, plant extracts, tissue samples, and the like. Examples of a “fluid biological sample” or “liquid biological sample” include blood, serum, plasma, sputum, lavage fluid (for example peritoneal lavage), cerebrospinal fluid, urine, vaginal discharge, semen, sweat, tears, saliva, and the like.

As used herein, the terms “blood”, “plasma”, and “serum” encompass fractions or processed portions thereof. Similarly, where a sample is taken from a biopsy, swab, smear, etc., the sample encompass a processed fraction or portion derived from the biopsy, swab, smear, etc.

In some examples, the sample may include a non-biological sample. In some examples, the non-biological sample may include any item that may contain the nucleic acid of interest. For example, the items may be a surface of an equipment, a laboratory bench, a public surface (such as, but not limited to, surface on an elevator/lift/doorknobs/toilet, surface on a public transport, surface of airport areas, surface of school areas, surface of shopping mall or supermarket areas, surface of restaurants/hawkers/cafes, and the like), frequently touched surfaces adjacent to patients in hospitals/clinics (such as, but not limited to, areas adjacent to or at the hospital bed, hospital/clinic waiting areas, quarantine rooms and the like).

In various embodiments, the method further comprises a step of quantifying the target nucleic acid.

In some examples, the target nucleic acid is quantified using one or more methods known in the art. In some examples, the target nucleic acid may be quantified using one or more methods such as, but not limited to, PCR-based detection systems (such as quantitative PCR (qPCR), ARMS-PCR, next generation sequencing, droplet digital PCR), UV absorbance with spectrophotometer, fluorescence dyes, gel electrophoresis (such as poly-acrylamide gel electrophoresis (PAGE)), capillary electrophoresis, microfluidic method, diphenylamine method, and the like.

In some examples, the method to quantify a nucleic acid may include qPCR.

In yet another aspect, there is provided a kit for detecting/identifying/determining a nucleic acid in a sample, the kit comprising one or more of the following: one or more primers/primer set (targeting a matched or a mismatched template) as described herein, and a proofreading polymerase (such as 3′-exonuclease proficient polymerase).

In various embodiments, the kit further comprises a control primer (such as a natural primer with no modifications), a proofreading polymerase, a primer with 2′-sugar modification, a control template, medium/buffer/solution, salt (such as MgSO₄), water (such as distilled water, RNase-free water and/or nuclease-free water); deoxynucleotide triphosphates (dNTPs) and a control dye (such as SYBR Green).

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g. primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′—OH group.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### Experimental Section

### Methods

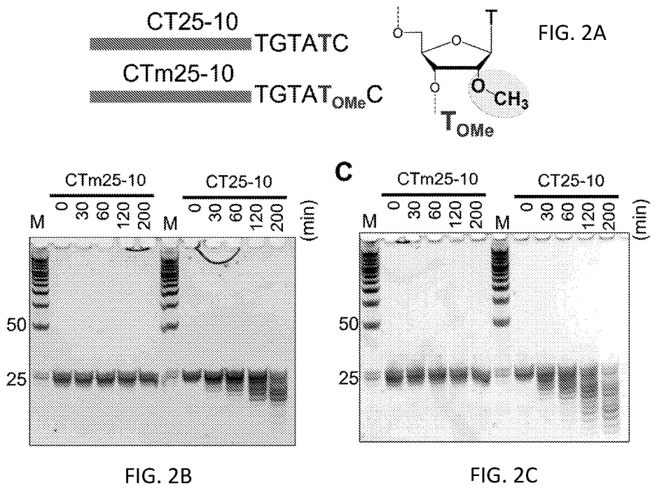

### Primer Stability Assays (FIG. 2)

DNA primers with or without a 2′-O-methylnucleoside (5′-CCGTCTCATGGATCAGGATTGTAXC-3′; X=T for CT25-10 (SEQ ID NO: 16), 2′-O-methyl-T for CTm25-10) (SEQ ID NO: 12) were diluted to 1 μM in a 100 μL solution (1×KOD-Plus-Ver.2 buffer with 1.5 mM MgSO₄, 0.02 U/μL KOD plus polymerase (pol) [TOYOBO] or 1×Pfu buffer including 2.0 mM MgSO₄, 0.025 U/μL Pfu polymerase (pol) [Promega]). The mixture was heated at 94° C. for 2 min, and then incubated at 68° C. Aliquots (10 μL) were removed at various time points from 0 to 200 min, and immediately mixed with 10 μL of denaturing solution (1×TBE containing 10 M urea and 0.025% BPB). Each sample (7 μL) was analyzed by 20% denaturing polyacrylamide gel electrophoresis, and the DNA band patterns were detected with a bio-imaging analyzer (Fuji Film LAS-4000) after SYBR Gold staining.

Real-Time qPCR (FIG. 3)

The PCR reaction (25 μL) was performed in 1×KOD-Plus-Ver.2 buffer with 1.5 mM MgSO₄, 0.2 mM dNTPs, 75,000-fold diluted SYBR Green I (Molecular Probes) and 0.02 U/μL KOD Plus pol, in the presence or absence of 0.2 μM template (69-mer, 3T-69G1 or 3T-69A2), using 0.3 μM/0.6 μM/1 μM each primer. PCR cycling parameters were 94° C. for 2 min, then 25 cycles of 15 s at 94° C., followed by 1 min/2 min/3 min/3.5 min at 68° C. on a CFX96 real-time PCR system (Bio Rad).

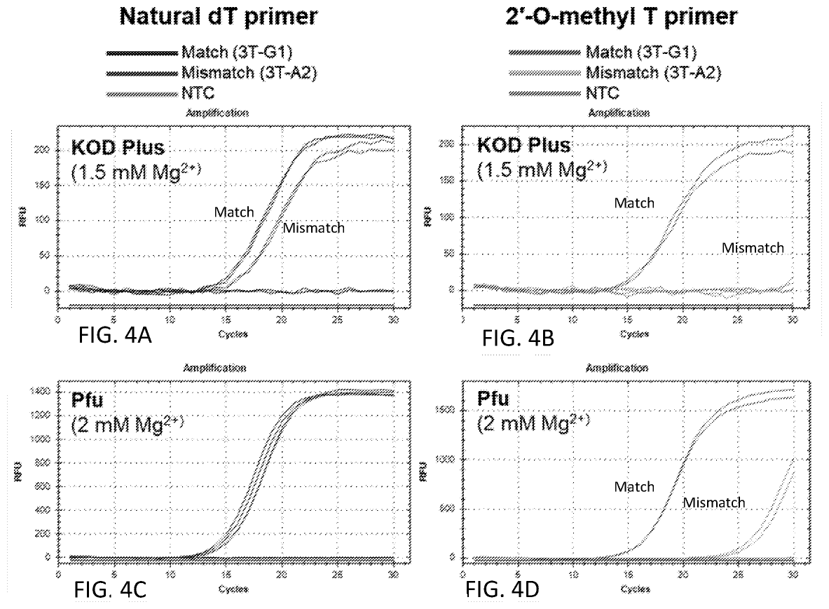

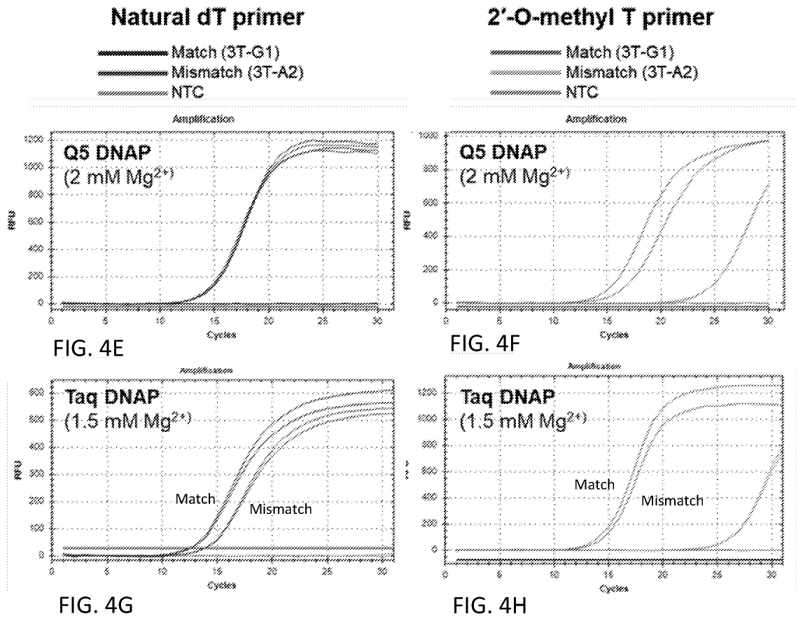

Real-Time qPCR (FIG. 4)

The PCR reaction (25 μL) was performed in 1× reaction buffer, with 1.5 or 2 mM MgSO₄, 0.2 mM dNTPs, 75,000-fold diluted SYBR Green I and 0.02 U/μL KOD Plus polymerase, 0.025 U/μL Pfu polymerase, 0.02 U/μL Q5 DNA polymerase (DNAP) (New England Biolabs/NEB) or 0.025 U/μL Taq DNAP (NEB), in the presence or absence of 0.2 μM template (69-mer, 3T-69G1 or 3T-69A2), using 0.3 μM of each primer. PCR cycling parameters were 94° C. for 2 min, then 30 cycles of 15 s at 94° C., followed by 3.5 min at 68° C., except for Taq DNAP with natural dT primers, with a 1 min eIongation step at 72° C.

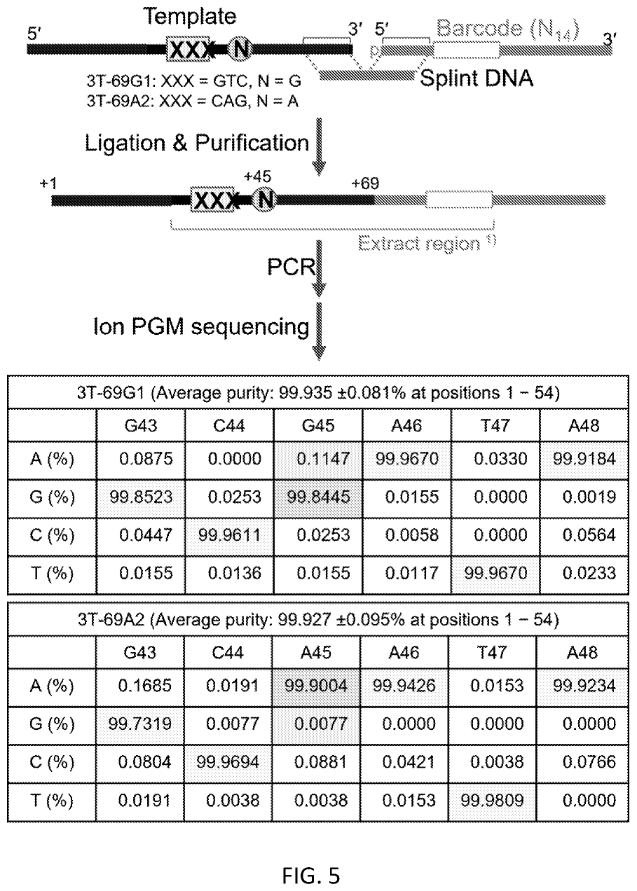

### Purity Analysis of Chemically Synthesized DNA Fragments (FIG. 5)

Barcode Adaptor DNA (600 pmol, 5′-CTCACTACGCNNNNNNNNNNNNNNCTTCGTTGAGAACCCACAATCTTAC-3′) (SEQ ID NO: 1) was phosphorylated by T4 polynucleotide kinase (NEB) in 1×T4 DNA ligation buffer (NEB) by an incubation at 37° C. for 30 min, and then the kinase was inactivated by heating at 65° C. for 20 min. Afterwards, the 20-mer Splint DNA (660 μmol, 5′-GCGTAGTGAGTCGGGTTATG-3′) (SEQ ID NO: 27) and 69-mer template DNA (600 μmol) were added to the solution, and the ligation reaction was performed in 1×T4 DNA ligation buffer with T4 DNA ligase at 16° C. for 30 min. The ligated products were purified by denaturing polyacrylamide gel electrophoresis. The ligated products (final conc.: 10 fmol/L) were used as the template for 30-cycle PCR (100 μL) with AccuPrime Pfx DNA pol using 25-mer forward and reverse primers (1 μM). The PCR products were subjected to deep sequencing analyses with the IonPGM system. The inventors of the present disclosure extracted the regions inside of the primers with the correct length (68-mer: Extracted region, (1)), and selected the reads (analyzed fragments/analyzed reads, (2)) with more than 2 counts under the same barcode sequence.

TABLE 4

| | Raw | Extracted | Analyzed fragments ²⁾ |
|---|---|---|---|
| Template | reads | reads ¹⁾ | (Analyzed reads) |
| 3T-69G1 | 737,603 | 526,374 | 51,448 |
| | | | (176,850) |
| 3T-69A2 | 581,815 | 380,618 | 26,113 |
| | | | (86,540) |

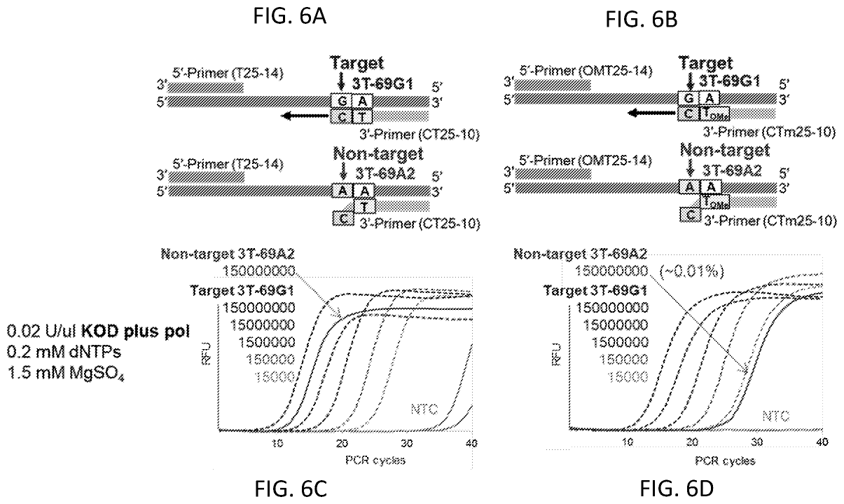

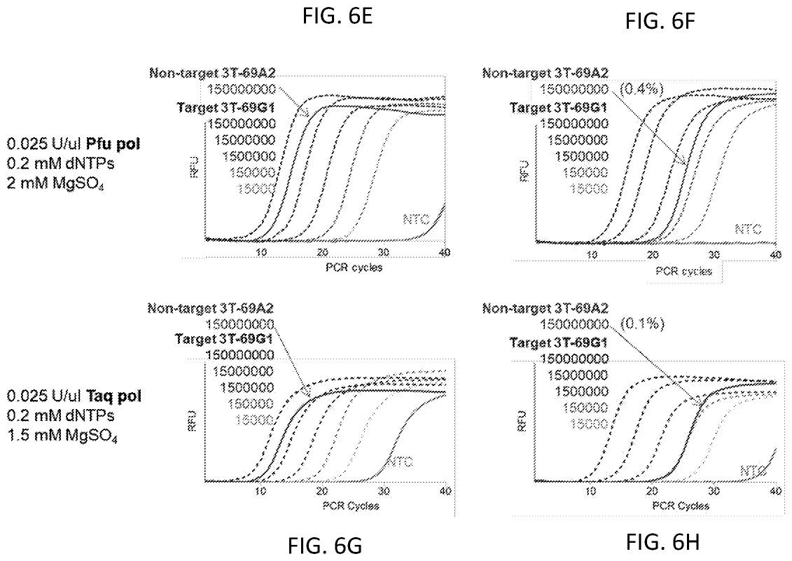

Real-Time qPCR for Mismatch Discrimination with Modified Primers (FIGS. 6-9)

The PCR reaction (25 μL) was performed in 1× reaction buffer with 1.5 or 2 mM MgSO₄, 0.2 mM dNTPs, diluted SYBR Green I, and either 0.02 U/μL KOD Plus polymerase, 0.025 U/μL Pfu polymerase, or 0.02 U/μL Taq DNAP (NEB), using various concentrations of the matched template (0.001, 0.01, 0.1, 1, or 10 μM) or the mismatched template (10 μM) and 0.3 μM of each modified primer. PCR cycling parameters were 94° C. for 2 min, then 40 cycles of 15 s at 94° C., and 3.5 min at 68° C.

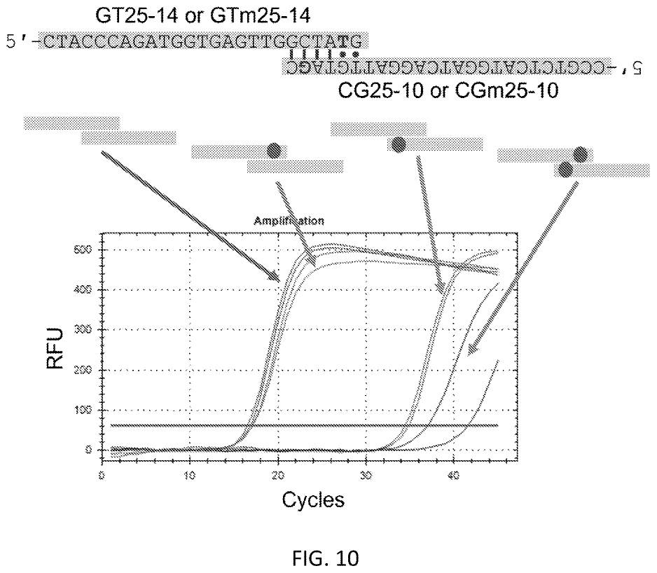

### Primer Dimer Formation Check (FIG. 10)

To examine artificial primer dimer formation during PCR, the inventors of the present disclosure performed PCR in 1×Pfu reaction buffer with 2.25 mM MgSO₄, 0.4 mM dNTPs, 75,000-fold diluted SYBR Green I and 0.025 U/μL Pfu polymerase using 1 μM of the non-modified and modified primer combinations. PCR cycling parameters were 94° C. for 2 min, then 45 cycles of 15 s at 94° C., and 3.5 min at 68° C.

To examine artificial primer dimer formation during PCR, the inventors of the present disclosure performed PCR in 1× reaction buffer with 1.5 mM MgSO₄, 0.2 mM dNTPs, diluted SYBR Green I, 0.02 U/μL KOD Plus polymerase, and 0.3 μM of each modified (or non-modified) primer. PCR cycling parameters were 94° C. for 2 min, then 45 cycles of 15 sec at 94° C. and 3.5 min at 68° C.

TABLE 5
DNA sequences used in this study

| | Sequence (5′ to 3′) |
|---|---|
| Primers | t = OMeT, a = OMeA, g = OMeG, c = OMeC, |
| forPCR | u = OMeU, I = LNA-T, f = 2′-fluoro-U |
| OMT25-10 | CCGTCTCATGGATCAGGATTGTAtC |
| | (SEQ ID NO: 12) |
| OMA25-10 | CCGTCTCATGGATCAGGATTGTAaC |
| | (SEQ ID NO: 13) |
| OMG25-10 | CCGTCTCATGGATCAGGATTGTAgC |
| | (SEQ ID NO: 14) |
| OMC25-10 | CCGTCTCATGGATCAGGATTGTAcC |
| | (SEQ ID NO: 15) |
| T25-10 | CCGTCTCATGGATCAGGATTGTATC |
| | (SEQ ID NO: 16) |
| OMU25-10 | CCGTCTCATGGATCAGGATTGTAuC |
| | (SEQ ID NO: 17) |
| LT25-10 | CCGTCTCATGGATCAGGATTGTAIC |
| | (SEQ ID NO: 18) |
| FU25-10 | CCGTCTCATGGATCAGGATTGTAfC |
| | (SEQ ID NO: 19) |
| TOMT25-10 | CCGTCTCATGGATCAGGATTGTAtT |
| | (SEQ ID NO: 20) |
| TT25-10 | CCGTCTCATGGATCAGGATTGTATT |
| | (SEQ ID NO: 21) |
| OMT25-14 | CTACCCAGATGGTGAGTTGGCTAtG |
| | (SEQ ID NO: 22) |
| T25-14 | CTACCCAGATGGTGAGTTGGCTATG |
| | (SEQ ID NO: 23) |
| OMU25-14 | CTACCCAGATGGTGAGTTGGCTAuG |
| | (SEQ ID NO: 24) |
| LT25-14 | CTACCCAGATGGTGAGTTGGCTAIG |
| | (SEQ ID NO: 25) |
| FU25-14 | CTACCCAGATGGTGAGTTGGCTAfG |
| | (SEQ ID NO: 26) |
| Templates | |
| for PCR | Sequence (5′ to 3′) |
| 3T-69G1 | CTACCCAGATGGTGAGTTGGCTATGTTGTAGTCTTGA |
| | CTATCGCGATACAATCCTGATCCATGAGACGG |
| | (SEQ ID NO: 4) |
| 3T-69A2 | CTACCCAGATGGTGAGTTGGCTATGTTGTACAGTTGA |
| | CTATCGCAATACAATCCTGATCCATGAGACGG |
| | (SEQ ID NO: 5) |
| 69(1) | CTACCCAGATGGTGAGTTGGCTATGTTGTAGTCTTGA |
| | ATATCGCGTTACAATCCTGATCCATGAGACGG |
| | (SEQ ID NO: 6) |
| 69(2) | CTACCCAGATGGTGAGTTGGCTATGTTGTAGTCTTGA |
| | ATATCGCATTACAATCCTGATCCATGAGACGG |
| | (SEQ ID NO: 7) |
| 69(3) | CTACCCAGATGGTGAGTTGGCTATGTTGTAGTCTTGA |
| | ATATCGCGCTACAATCCTGATCCATGAGACGG |
| | (SEQ ID NO: 8) |
| 69(4) | CTACCCAGATGGTGAGTTGGCTATGTTGTAGTCTTGA |
| | ATATCGCACTACAATCCTGATCCATGAGACGG |
| | (SEQ ID NO: 9) |
| 69(5) | CTACCCAGATGGTGAGTTGGCTATGTTGTAGTCTTGA |
| | ATATCGCGGTACAATCCTGATCCATGAGACGG |
| | (SEQ ID NO: 10) |
| 69(6) | CTACCCAGATGGTGAGTTGGCTATGTTGTAGTCTTGA |
| | ATATCGCAGTACAATCCTGATCCATGAGACGG |
| | (SEQ ID NO: 11) |
| Fragments | |
| for DNA | |
| purity | |
| analysis | Sequence (5′ to 3′) |
| BacoreAda | CTCACTACGCNNNNNNNNNNNNNNCTTCGTTGAGAAC |
| ptor49 | CCACAATCTTAC |
| | (SEQ ID NO: 1) |
| Splint20 | GCGTAGTGAGCCGTCTCATG |
| | (SEQ ID NO: 2) |
| Primer | GTAAGATTGTGGGTTCTCAACGAAG |
| 25mer | (SEQ ID NO: 3) |

### Results

DNA Primer Stabilization by Introducing a 2′-O-Methylnucleoside at the Penultimate Position from the 3′-Terminus

DNA primers (25-mer) with or without a 2′-O-methylnucleoside at the second position from the 3′-terminus (FIG. 2A) were chemically synthesized by the standard phosphoramidite method, using commercially available amidite reagents of the 2′-O-methylnucleosides of A, G, C, T, and U. The primers (CT25m-10 and CT25-10) were incubated at 68° C. with KODplus or Pfu DNA polymerases, and the digestions were analyzed by denaturing gel electrophoresis. Most of the unmodified DNA primer (CT25-10) was degraded after 200 min by these two polymerases (FIGS. 2B and 2C). In contrast, no digestion of the modified DNA primer (CTm25-10) was observed in the incubation for up to 200 min (FIGS. 2B and 2C).

### Optimization of the Polymerase Chain Reaction (PCR) Amplification Using 2′-O-Methylnucleoside Primers and Proofreading DNA Polymerases

FIGS. 3A to 3C show PCR amplification efficiency using KODplus DNA polymerase (exo+) and modified DNA primers containing a 2′-O-methylnucleoside at the penultimate position from the 3′-end. To optimize the PCR conditions using modified primers containing 2′-O-methylnucleosides at the penultimate position from the 3′ end (OMe-primer), the inventors of the present disclosure first determined the optimal primer concentrations using KOD Plus DNA polymerase (exo+) (FIGS. 3A and 3B). Generally, a 1 μM concentration of primers is used for PCR. However, the inventors of the present invention found that 1 μM of OMe-primers reduced the PCR amplification efficiency, as compared to that using the unmodified primers. By reducing the OMe-primer concentration to 0.3 μM, the amplification efficiency was recovered to a level as high as that of the conventional method using 1 μM unmodified primers with the G-C matched template (3T-69G1). The OMe-primers completely prevented the amplification with the A-C mismatched template (3T-69A2). In contrast, the conventional method using 1 M unmodified primers also amplified the A-C mismatched template. Next, the inventors of the present disclosure optimized the PCR eIongation time using 0.3 μM OMe primers and determined that a duration Ionger than 3 min was required for efficient amplification using the OMe primers, which was as high as that using 1 μM of the unmodified primers (FIG. 3C).

FIGS. 4A to 4G show comparison of the method of the present disclosure using modified primers (FIGS. 4B, 4D, 4F, and 4H) with the conventional method (FIGS. 4A, 4C, 4E, and 4G). Three exo⁺ (KOD Plus, Pfu, and Q5) and one exo-(Taq) DNA polymerases were examined for the SNP detection with unmodified and OMe-primers (FIG. 4). Among these polymerases, KOD Plus DNA polymerase exhibited the highest fidelity for the SNP detection between the G-C match and A-C mismatch template-primer pairs (FIG. 4B). Under these conditions, the amplification of the A-C mismatch template-primer (OMe) pair was not observed by 30-cycle PCR. Other polymerases, even Taq DNA polymerase, exhibited higher fidelity with the OMe-primers, as compared to that obtained with the unmodified primers.

### Determination of the Purity of Chemically Synthesized DNAs

To quantitatively analyze the discrimination of the A-C or G-T mismatch pairs from the respective matched pairs of G-C or A-T, the inventors of the present disclosure chemically synthesized two DNA templates, 3T-69G1 and 3T-69A2, by the conventional phosphoramidite method using an automated DNA synthesizer. Chemically synthesized DNA fragments generally contain a tiny number of DNAs with failure sequences, because of the intrinsic mechanical issues depending on the type of DNA synthesizer machine. Thus, prior to the SNP detection experiments, the inventors of the present disclosure determined the purity of the synthesized DNA templates by barcode sequencing, using a next generation sequencer, the Ion personal genome machine (PGM) system (FIG. 5).

Each of the DNA fragments, 3T-69G1 and 3T-69A2, was ligated with a 49-mer DNA fragment containing N14 barcodes with a splint DNA (20-mer), and the ligated full-length DNA (around 1 amol) was amplified by PCR for deep sequencing. The barcode primers recognize and remove the mutations in each amplified DNA resulting from the PCR and sequencing procedures. The inventors of the present disclosure analyzed 51,448 sequences for 3T-69G1 and 26, 113 sequences for 3T-69A2 as PCR/sequencing error-free sequences. Among the sequences, 3T-69G1 contained 99.8445% of the correct G and 0.1147% of the incorrect A at position 45 (SNP position), and 3T-69A2 contained 99.9004% of the correct A and 0.0077% of the incorrect G at position 45. Accordingly, analyses of the results obtained by SNP experiments should include considerations of these impurities in each chemically synthesized DNA fragment.

### Quantitative SNP Detection by the Combination of OMe-Primers and Exo+ DNA Polymerases.

FIGS. 6A to 6G show the comparison of the A-C mismatch discrimination by the method of the present disclosure using modified primers (FIGS. 6B, 6D, 6F, and 6H) with that by the conventional method (FIGS. 6A, 6C, 6E, and 6G). To determine the detection sensitivity between the matched G-C and mismatched A-C pairs in the template-primer hybrid, the inventors of the present disclosure performed the qPCR amplification of a series of different amounts (1.5×10⁴ to ×10⁸ molecules) of the matched target, 3T-69G1, and 1.5×10⁸ molecules of the mismatched non-target, 3T-69A2, using the unmodified (FIG. 6A) or OMe-primers (FIG. 6B). When using the combination of KOD plus DNA polymerase and OMe-primers, the amplification efficiency of 1.5×10⁸ molecules of the mismatched non-target, 3T-69A2, was as low as that of 1.5×10⁴ of the matched target, 3T-69G1, indicating that around 0.01% of the target DNA in the one-base mismatched DNA can be detected (FIG. 6D). Furthermore, since 3T-69A2 contains 0.0077% of G at the SNP position, the mis-amplification by the A-C mismatch might be much lower (<0.003%) when using the OMe-primers.

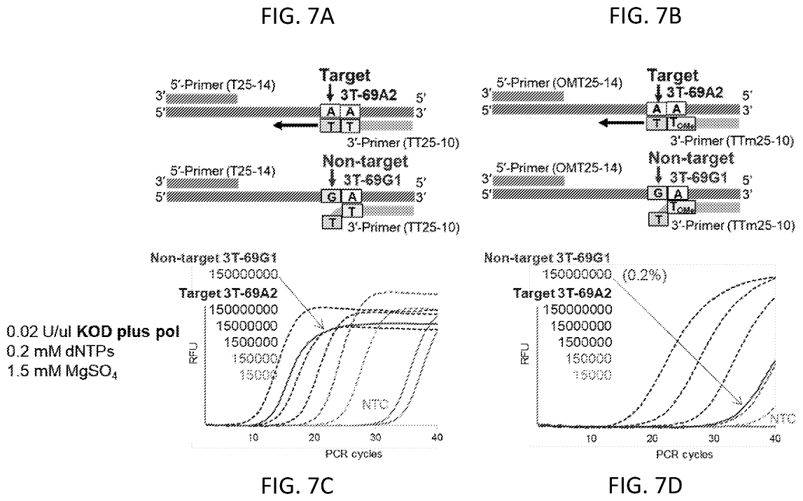

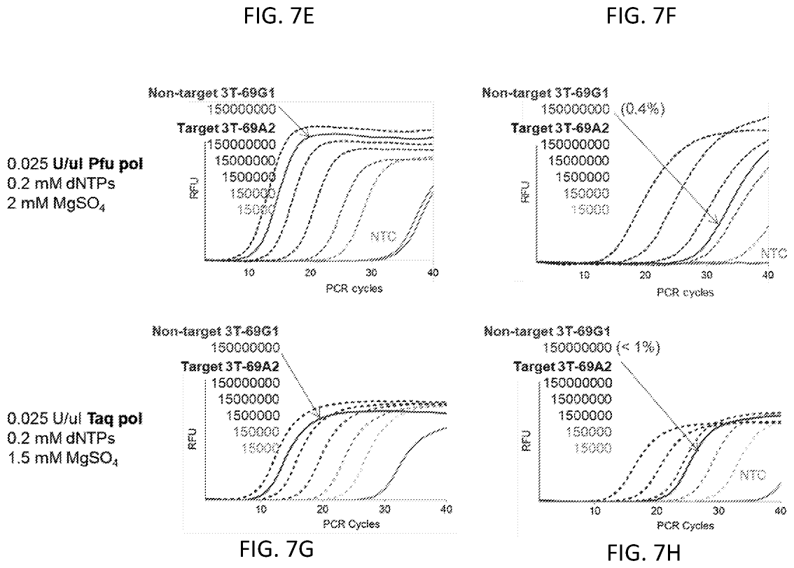

Next, the inventors of the present disclosure examined the amplification sensitivity of the G-T mismatch (FIG. 7A to 7G). FIGS. 7A to 7G includes the comparison of the G-T mismatch discrimination by the method of the present disclosure using modified primers (FIGS. 7B, 7D, 7F, and 7H) with that by the conventional method (FIGS. 7A, 7C, 7E, and 7G). In general, it is difficult to discriminate the G-T mismatch by conventional PCR methods, due to the wobble base pair formation. Thus, the conventional method using the unmodified primers and Taq DNA polymerase efficiently amplified the non-target DNA, to a level as high as that of the target DNA (FIG. 7G). Even in this case, the combination of the OMe-primers and KOD plus DNA polymerase exhibited high fidelity, and the amplification efficiency of the mismatched non-target, 3T-69G1, was 0.2% as compared to that of the matched target, 3T-69A2, (FIG. 7D). Similar to the A-C mismatch experiments, 3T-69G1 contains 0.1147% of A at the SNP position, and thus the mis-amplification rate by the OMe-primer and KOD combination might be lower than 0.1%.

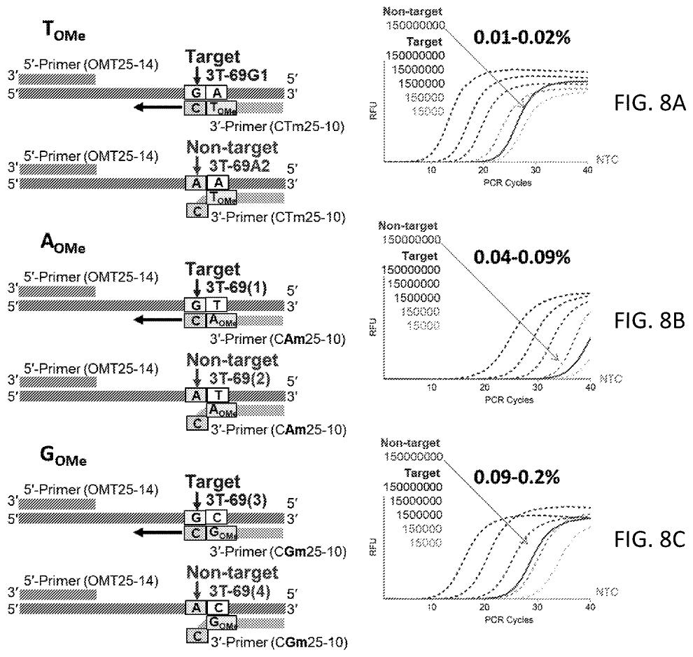

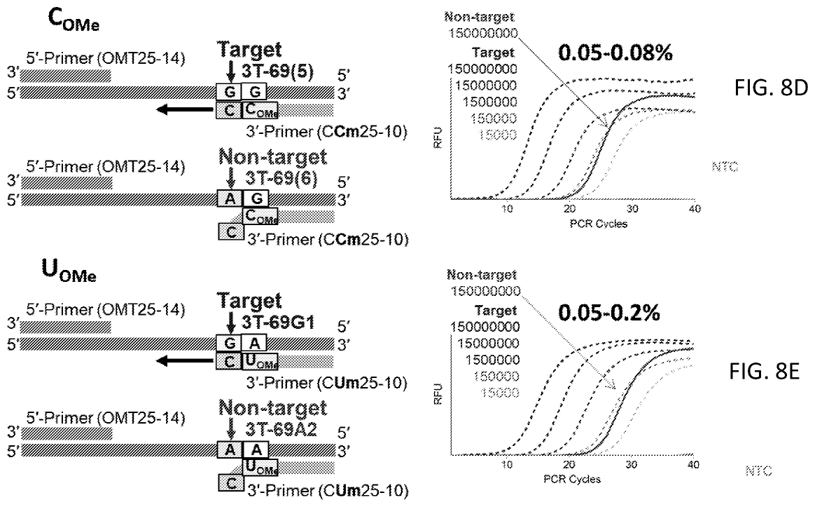

In these experiments, the inventors of the present disclosure used OMe-primers containing 2′-O-methyl-thymidine (T_{OMe}). Next, the inventors of the present disclosure examined the effectiveness of the other 2′-O-methyl-nucleosides of adenosine (A_{OMe}), guanosine (G_{OMe}), cytidine (C_{OMe}), and uridine (V_{OMe}) as the OMe-primers (FIG. 8). Although T_{OMe} exhibited the highest specificity, those of the reactions with the 2′-O-methyl-nucleosides of other bases were also high (0.04-0.2%) with the target DNAs, using KOD plus polymerase.

The inventors of the present disclosure also examined the SNP detection using primers with other modifications, such as 2′-fluoro (F) and locked nucleic acid (LNA), in combination with KOD plus DNA polymerase (FIG. 9). The 2′-fluoro modification of U (U_{F} in FIG. 9) was not effective for the SNP detection, as compared to the 2′-O-methyl modification of U (U_{OMe} in FIG. 8). As previously reported, the high specificity of the LNA modification was confirmed, although the sensitivity of the SNP detection was slightly lower than that of the 2′-O-methyl modification (T_{OMe} and T_{LNA} in FIG. 9).

### Prevention of Primer-Dimer Formation by OMe-Primers in PCR

In addition to the improved sensitivity of the method of the present disclosure, the inventors of the present disclosure found that the OMe-primers prevent the artefactual primer dimer formation during PCR. The primer dimer formation at the 3′-regions in each of the forward and reverse primers causes false-positive outcomes in qPCR. The inventors of the present disclosure examined the primer dimer amplification by PCR, using KOD plus DNA polymerase with combinations of the unmodified and OMe-primers. For these primer-dimer experiments, the forward and reverse primers were designed to have highly complementary sequences at their 3′-ends (FIG. 10). Therefore, the unmodified primers efficiently formed the primer dimers, and the false-positive amplification was observed after 10 cycles of PCR.

In contrast, by using forward and reverse OMe-primers, the primer dimer amplification was significantly reduced. The combination of GT25-14 and CGm25-10 also effectively prevented the primer dimer formation. However, the primer dimers were efficiently produced when using the GTm25-14 and CG25-10 pair, indicating that the primer dimer formation by the cognate natural base pairings between primers is more effective, as compared to that of the G-T wobble pair.

The inventors of the present disclosure developed a high-fidelity PCR system, using 3′-exonuclease-proficient DNA polymerases and OMe-primers containing one 2′-O-methylribonucleoside at the penultimate position from their 3′-ends, for quantitative SNP detection. The OMe-primers are resistant to degradation by the exonuclease-proficient DNA polymerases, and the primer-polymerase combination efficiently recognizes the mis-match pair at the 3′-ends of the primers by PCR. Although any sequence contexts can be used with this method, 2′-O-methyl thymidine exhibited the highest fidelity as compared to the other bases and the other modifications, such as 2′-fluoro and LNA nucleosides. The OMe-primers also prevented primer-dimer formation in PCR. This simple method will facilitate the highly specific detection of SNPs, viral and bacterial variants, and genomic mutations by qPCR.

### DETAILED DESCRIPTION OF FIGURES

Example embodiments of the disclosure will be better understood and readily apparent to one of ordinary skill in the art from the following discussions and if applicable, in conjunction with the figures. Example embodiments are not necessarily mutually exclusive as some may be combined with one or more embodiments to form new exemplary embodiments. The example embodiments should not be construed as limiting the scope of the disclosure.

FIGS. 1A to 1D show schematic diagrams of conventional allele-specific qPCR method using exo-DNA polymerase.

FIGS. 1E to 1G show schematic diagrams of the new method using 2′-O-methyl-modified primers in combination with exo+ DNA polymerase for quantitative SNP/mutant detection.

FIG. 2A. shows the sequence and structure of modified DNA primer containing a 2′-O-methylnucleoside at the penultimate position from the 3′-terminus.

FIG. 2B shows blots of stability tests of the modified and unmodified primers with KOD Plus DNA polymerase.

FIG. 2C shows blots of stability tests of the modified and unmodified primers with Pfu DNA polymerases.

FIG. 3A shows sequences of DNA templates and modified or unmodified PCR primers.

FIG. 3B shows plots with amplification efficiencies of PCR with varied concentrations of the modified primers.

FIG. 3C shows plots with amplification efficiencies of PCR with varied elongation periods.

FIGS. 4A and 4B show plots of experiments that were performed using KOD plus DNA polymerase with exo+ with matched and unmatched templates.

FIGS. 4C and 4D show plots of experiments that were performed using Pfu polymerase with exo+ with matched and unmatched templates.

FIGS. 4E and 4F show plots of experiments that were performed using Q5 polymerase with exo+ with matched and unmatched templates.

FIGS. 4G and 4H show plots of experiments that were performed using Taq polymerase with exo− with matched and unmatched templates.

FIG. 5 shows a schematic diagram with determination of the purities of the chemically synthesized DNA fragments, 3T-69G1 and 3T-69A2, by barcode sequencing.

FIGS. 6A and 6B show diagrams of modified and unmodified primers with their matched target and unmatched non-target templates.

FIGS. 6C and 6D show plots with experiments that were performed using KOD plus DNA polymerase with exo+.

FIGS. 6E and 6F show plots with experiments that were performed using Pfu DNA polymerase with exo+ with matched target and unmatched non-target templates.

FIGS. 6G and 6H show plots with experiments that were performed using Taq polymerase with exo− with matched target and unmatched non-target templates.

FIGS. 7A and 7B show diagrams of modified and unmodified primers with their matched target and unmatched non-target templates.

FIGS. 7C and 7D show plots with experiments that were performed using KOD plus DNA polymerase with exo+ with matched target and unmatched non-target templates.

FIGS. 7E and 7F show plots with experiments that were performed using Pfu DNA polymerase with exo+ with matched target and unmatched non-target templates.

FIGS. 7G and 7H show plots with experiments that were performed using Taq DNA polymerase with exo− with matched target and unmatched non-target templates.

FIG. 8 shows plots that measure sensitivities of SNP detection by qPCR using 2′-O-methylnucleosides of different bases with KOD plus DNA polymerase.

FIG. 9A to 9C show plots that measure sensitivities of the SNP detection by qPCR using 2′-O-methyl-thymidine, 2′-fluoro modification of U (U_{F}) and LNA with KOD plus DNA polymerase respectively.

FIG. 10 shows plots of the 2′-O-methyl modification effectively preventing primer-dimer formation in PCR with KOD plus DNA polymerase.

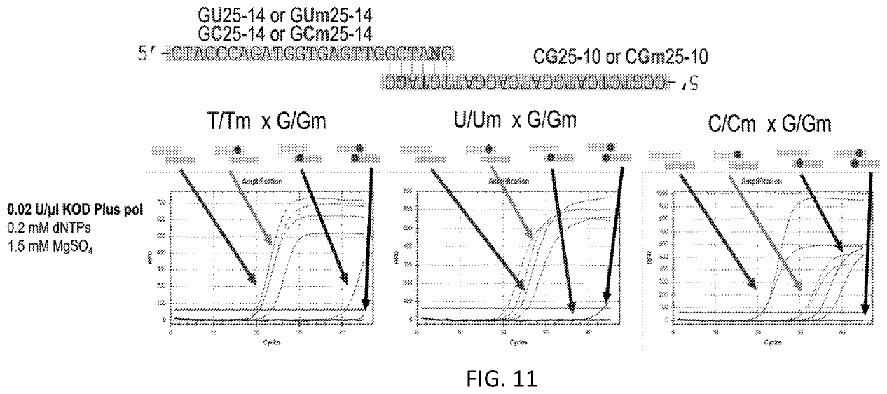

FIG. 11 shows plots of the 2′-O-methyl modification effectively preventing primer-dimer formation in PCR with KOD plus DNA polymerase.

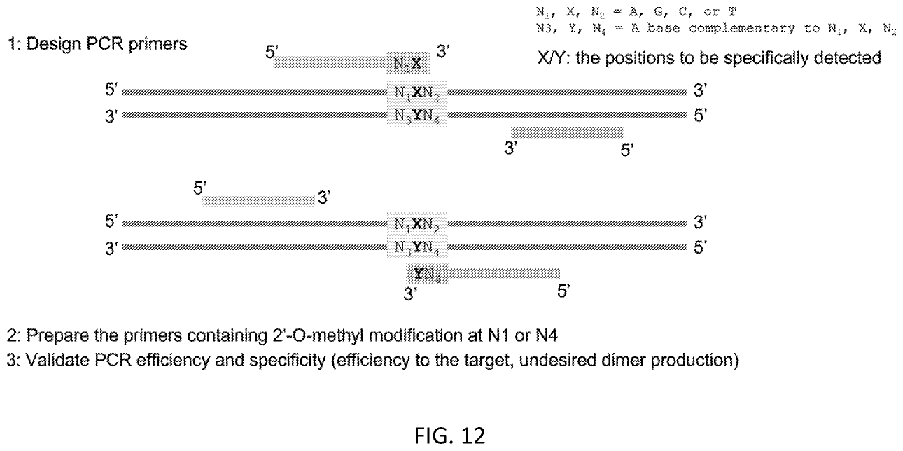

FIG. 12 shows a diagram on the design of specific primers to detect one-nucleotide difference in PCR.

### APPLICATIONS

Embodiments as disclosed herein provide methods of detecting the presence of a nucleic acid in a sample, a method of identifying a disease in a subject and a kit for detecting a nucleic acid in a sample.

Advantageously, the present invention provides a high fidelity and high sensitivity amplification method using the combination of modified primers and proofreading polymerase by increasing the stability of the primers against 3′ exonuclease activity of high-fidelity polymerases.

More advantageously, the present invention facilitates highly specific detection of single nucleotide polymorphisms (SNP) in a sample (such as SNP detection of genome for therapeutics, viral mutation detection in infectious disease), viral and bacterial variants, and genomic mutations.

Even more advantageously, the present invention is simple and cost effective compared to other primer modifications known in the art.

Even more advantageously, the present invention significantly reduces primer dimer formation during amplification reaction.

Even more advantageously, the present invention provides an authentication method using DNA ink, since it requires specific priming and amplification even in the presence of large amounts of backgrounds.

Even more advantageously, the present invention provides diagnostics for cancer.

Even more advantageously, the present invention provides another PCR-based detection system.

Even more advantageously, the present invention can be extended to include visual detection of the amplified DNA by polymerase chain reaction using a genetic alphabet expansion system; or visible PCR detection of quinolone-resistant bacteria (such as SNP of quinolone resistant bacteria mutants).

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A method of detecting the presence of a nucleic acid in a sample,
the method comprises performing an amplification reaction on the sample in the presence of a proofreading polymerase and a primer with a 2′-sugar modification.

2. The method according to claim 1, wherein the proofreading polymerase is an exonuclease proficient polymerase.

3. The method according to claim 1, wherein the proofreading polymerase is a 3′-exonuclease proficient polymerase.

4. The method according to claim 1, wherein the 2′-sugar modification is at locations in the primer comprising the 5′ end of the primer, the middle of the primer, or the 3′ end of the primer.

5. The method according to claim 1, wherein the 2′-sugar modification is at the 3′ end of the primer.

6. The method according to claim 1, wherein the 2′-sugar modification is at a position that is two residues from the 3′ end of the primer.

7. The method according to claim 1, wherein the 2′-sugar modification is at penultimate position of the 3′ end of the primer.

8. The method according to claim 1, wherein the primers with 2′-sugar modification comprises 2′-O-methyl-ribonucleosides modification or 2′-fluoro.

9. The method according to claim 1, wherein the 2′-sugar modification is one modification selected from the group consisting of 2′-O-methyl C primer/C_{OME}, 2′-O-methyl T primer/T_{OME}, 2′-O-methyl U primer/U_{OME}, 2′-O-methyl G primer/G_{OME}, and 2′-O-methyl A primer/A_{OME}.

10. The method according to claim 1, wherein the primer comprises 15 to 30 nucleic acid residues.

11. The method according to claim 1, wherein the method comprises detecting the presence of a nucleotide mismatch or nucleotide polymorphism.

12. The method according to claim 1, wherein the method comprises detecting the presence of a single nucleotide polymorphism (SNP).

13. The method according to claim 1, wherein the method further comprises the step of quantification of the nucleic acid detected in the sample.

14. The method according to claim 1, wherein the method comprises a method of genome detection for therapeutics, or authentication methods.

15. A method of identifying a disease in a subject, comprising detecting the presence of a nucleic acid fragment by performing an amplification reaction on a sample obtained from the subject in the presence of a proofreading polymerase and a primer with a 2′-sugar modification.

16. The method according to claim 15, wherein the nucleic acid fragment/template comprises a sequence selected from the group listed in Table 2 or a part thereof.

17. The method according to claim 15, wherein the primers comprise a sequence selected from the group listed in Table 3 or a part thereof.

18. The method according to claim 15, wherein the sample is a biological sample or a clinical sample.

19. The method according to claim 15, wherein the method further comprises a step of quantifying the target nucleic acid.

20. A kit for detecting a nucleic acid in a sample, the kit comprising one or more of the following: one or more primers/primer set comprising a primer with 2′-sugar modification and a proofreading polymerase.


SEQUENCE LISTING (27 sequences)

SEQ ID NO: 1 - DNA, 49 bp, synthetic construct
  source 1..49: mol_type = other DNA
ctcactacgcnnnnnnnnnnnnnncttcgttgagaacccacaatcttac

SEQ ID NO: 2 - DNA, 20 bp, synthetic construct
  source 1..20: mol_type = other DNA
gcgtagtgagccgtctcatg

SEQ ID NO: 3 - DNA, 25 bp, synthetic construct
  source 1..25: mol_type = other DNA
gtaagattgtgggttctcaacgaag

SEQ ID NO: 4 - DNA, 69 bp, synthetic construct
  source 1..69: mol_type = other DNA
ctacccagatggtgagttggctatgttgtagtcttgactatcgcgatacaatcctgatccatgagacgg

SEQ ID NO: 5 - DNA, 69 bp, synthetic construct
  source 1..69: mol_type = other DNA
ctacccagatggtgagttggctatgttgtacagttgactatcgcaatacaatcctgatccatgagacgg

SEQ ID NO: 6 - DNA, 69 bp, synthetic construct
  source 1..69: mol_type = other DNA
ctacccagatggtgagttggctatgttgtagtcttgaatatcgcgttacaatcctgatccatgagacgg

SEQ ID NO: 7 - DNA, 69 bp, synthetic construct
  source 1..69: mol_type = other DNA
ctacccagatggtgagttggctatgttgtagtcttgaatatcgcattacaatcctgatccatgagacgg

SEQ ID NO: 8 - DNA, 69 bp, synthetic construct
  source 1..69: mol_type = other DNA
ctacccagatggtgagttggctatgttgtagtcttgaatatcgcgctacaatcctgatccatgagacgg

SEQ ID NO: 9 - DNA, 69 bp, synthetic construct
  source 1..69: mol_type = other DNA
ctacccagatggtgagttggctatgttgtagtcttgaatatcgcactacaatcctgatccatgagacgg

SEQ ID NO: 10 - DNA, 69 bp, synthetic construct
  source 1..69: mol_type = other DNA
ctacccagatggtgagttggctatgttgtagtcttgaatatcgcggtacaatcctgatccatgagacgg

SEQ ID NO: 11 - DNA, 69 bp, synthetic construct
  source 1..69: mol_type = other DNA
ctacccagatggtgagttggctatgttgtagtcttgaatatcgcagtacaatcctgatccatgagacgg

SEQ ID NO: 12 - DNA, 25 bp, synthetic construct
  source 1..25: mol_type = other DNA
ccgtctcatggatcaggattgtatc

SEQ ID NO: 13 - DNA, 25 bp, synthetic construct
  source 1..25: mol_type = other DNA
ccgtctcatggatcaggattgtaac

SEQ ID NO: 14 - DNA, 25 bp, synthetic construct
  source 1..25: mol_type = other DNA
ccgtctcatggatcaggattgtagc

SEQ ID NO: 15 - DNA, 25 bp, synthetic construct
  source 1..25: mol_type = other DNA
ccgtctcatggatcaggattgtacc

SEQ ID NO: 16 - DNA, 25 bp, synthetic construct
  source 1..25: mol_type = other DNA
ccgtctcatggatcaggattgtatc

SEQ ID NO: 17 - DNA, 25 bp, synthetic construct
  source 1..25: mol_type = other DNA
  modified_base 24: mod_base = um
ccgtctcatggatcaggattgtanc

SEQ ID NO: 18 - DNA, 25 bp, synthetic construct
  source 1..25: mol_type = other DNA
  modified_base 24: mod_base = OTHER; note = locked nucleic acid T (LNA-T)
ccgtctcatggatcaggattgtanc

SEQ ID NO: 19 - DNA, 25 bp, synthetic construct
  source 1..25: mol_type = other DNA
  modified_base 24: mod_base = OTHER; note = 2'-fluoro-U
ccgtctcatggatcaggattgtanc

SEQ ID NO: 20 - DNA, 25 bp, synthetic construct
  source 1..25: mol_type = other DNA
  modified_base 24: mod_base = tm
ccgtctcatggatcaggattgtant

SEQ ID NO: 21 - DNA, 25 bp, synthetic construct
  source 1..25: mol_type = other DNA
ccgtctcatggatcaggattgtatt

SEQ ID NO: 22 - DNA, 25 bp, synthetic construct
  source 1..25: mol_type = other DNA
  modified_base 24: mod_base = tm
ctacccagatggtgagttggctang

SEQ ID NO: 23 - DNA, 25 bp, synthetic construct
  source 1..25: mol_type = other DNA
ctacccagatggtgagttggctatg

SEQ ID NO: 24 - DNA, 25 bp, synthetic construct
  source 1..25: mol_type = other DNA
  modified_base 24: mod_base = um
ctacccagatggtgagttggctang

SEQ ID NO: 25 - DNA, 25 bp, synthetic construct
  source 1..25: mol_type = other DNA
  modified_base 24: mod_base = OTHER; note = locked nucleic acid T (LNA T)
ctacccagatggtgagttggctang

SEQ ID NO: 26 - DNA, 25 bp, synthetic construct
  source 1..25: mol_type = other DNA
  modified_base 24: mod_base = OTHER; note = 2'-fluoro-U
ctacccagatggtgagttggctang

SEQ ID NO: 27 - DNA, 20 bp, synthetic construct
  source 1..20: mol_type = other DNA
gcgtagtgagtcgggttatg